(19) United States
(12) Patent Application Publication
(10) Pub. No.: US 2006/0199215 A1
(43) Pub. Date: Sep. 7, 2006

(54) Waste water inspecting agent and waste water inspecting apparatus using the same

(75) Inventors: Takatoshi Kinoshita, Aichi (JP); Shintaro Washizu, Shizuoka (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/429,180

(22) Filed: May 8, 2006

Related U.S. Application Data
(62) Division of application No. 10/103,884, filed on Mar. 25, 2002.

(30) Foreign Application Priority Data
Mar. 23, 2001 (JP) 2001-86318

(51) Int. Cl. C12Q 1/68; C12M 1/34

(52) U.S. Cl. 435/6; 435/287.2

(57) ABSTRACT

A waste water inspecting agent is used as an additive to a specimen solution and comprises a rod-shaped body and a capturing structured element bonded to the rod-shaped body which specifically captures an object to be captured in the specimen solution.

### BACKGROUND OF THE INVENTION

1. Field of the Invention

The present invention relates to a waste water inspecting agent by which safety of waste water effluent from factories and the like may be easily and surely inspected and also to a waste water inspecting apparatus using the same.

2. Description of the Related Art

In recent years, an increase in industrial waste discharged from factories, enterprises, and the like has become a social problem. Among those industrial wastes, waste water discharged in a large amount requires urgent and effective countermeasures to be taken in order to effectively reduce pollution.

Example of such industrial waste water include washed and rinsed liquids from a washing step of metal, glass, resin and printed circuit board, waste water from the developing generated from a plating step and the like. On the other hand, with regard to a domestic wastewater, examples include food waste water discharged from restaurants and the like. All of them contain organic substances as components and the substances are recycled back to nature by methods using membrane, activated charcoal, microbes and the like.

Conventionally, in inspecting the waste waters or particularly in measuring heavy metals, the sample is extracted with a solvent or evaporated to concentrate under an acidic condition and then the concentration of the aimed element in the inspection solution is determined by means of atomic absorption spectrometry.

In the atomic absorption spectrometry, however, the light source is different for each of the aimed elements and a pretreatment operation and analytic method are different as well. Accordingly, there are problems in that the operation is generally troublesome, time consuming until analytical result is obtained and moreover, automated measurement is difficult.

Consequently, there has been a strong demand for safety of waste water discharged from factories, and for such waste water to be easily and quickly inspected.

Thus, an object of the present invention is to provide a waste water inspecting agent by which heavy metals, harmful organic compounds, agricultural chemicals, genetic recombinant cells, and the like contained in waste water from factories, and the like may be quickly and easily inspected and also to provide a waste water inspecting apparatus using the same.

The waste water inspecting apparatus of the present invention is used as an additive to a specimen solution and comprises a rod-shaped body and a capturing structured element which specifically captures an object to be captured which is captured to the rod-shaped body in the specimen solution, thereby Heavy metals, harmful organic compounds, agricultural chemicals, and gene recombinated cells may be detected swiftly and simply.

The first aspect of the waste water inspecting apparatus of the present invention is that it has a rod-shaped body having a length of 810 mm or less and a capturing structured element specifically capturing, by bonding to the rod-shaped body, an object to be captured contained in the specimen solution. The waste water inspecting apparatus also reflects an incident light as colored interference light by aligning in a film-like shape and is provided with an adding means for contacting the waste water inspecting agent to a sample to be examined and a wavelength measuring means for measuring the change in the wavelength brought out by the light reflection as colored interference light of the film-shaped waste water inspecting agent which captures the object to be captured.

The waste water inspecting agent aligned in a film-like shape reflecting the incident light as colored interference light on the basis of a multi-layer thin film interference theory which is a basic principle of color foramtion of the scaly powder of the wings of a Morpho butterfly. When the change in wavelength based on light reflectance as colored interference light by changes in length or refractive index upon the specific capturing of the object to be captured by the film like waste water inspecting agent is measured, presence of the captured object may be inspected and monitoring of waste treatment is possible.

The second aspect of the waste water inspecting apparatus of the present invention is that it has a rod-shaped body and a capturing structured element which specifically captures, by bonding to the rod-shaped body, an object to be captured contained in the specimen solution and is provided with a biosensor where a waste water inspecting agent which is amphiphilic is adhered and bonded in a film-like shape to a quartz oscillator or a surface acoustic wave (SAW) element, an oscillation circuit where a mass change or a viscoelasticity change when the object to be captured is captured by the biosensor is oscillated as a frequency, and a frequency counter where the frequency of the oscillation oscillated from the oscillation circuit is measured.

As a result, changes in mass or changes in viscoelasticity when the capturing structured element of the waste water inspecting agent constituting the biosensor specifically captures the object to be captured may be detected as a frequency under high sensitivity in a short time.

### BRIEF DESCRIPTION OF THE DRAWINGS

FIG. 1 is a schematic view of a waste water inspecting agent relating to one example of the present invention.

FIG. 2 is a view for explaining a principle of light reflection of an incident light as colored interference light.

FIG. 3 is a typical view to explain the principle of light reflection of the incident light as colored interference light.

FIG. 4 is a schematic view for showing a formation of a monomolecular film by a functional molecule of the present invention.

FIG. 5 is a schematic view for showing an example of an amphiphilic functional molecule aligned on water (aqueous phase).

FIG. 6 is a schematic view for showing an example of an amphiphilic functional molecule vertically aligned on water (aqueous phase).

FIGS. 7A and 7B are example views of a quartz oscillator in which FIG. 7A is a plan view and FIG. 7B is a front view.

FIG. 8 is a schematic view which shows an example of a waste water inspecting apparatus.

FIG. 9 is a schematic plan view showing a surface acoustic wave (SAW) element.

### DESCRIPTION OF THE PREFERRED EMBODIMENTS

Hereinafter, the present invention will be described in more detail.

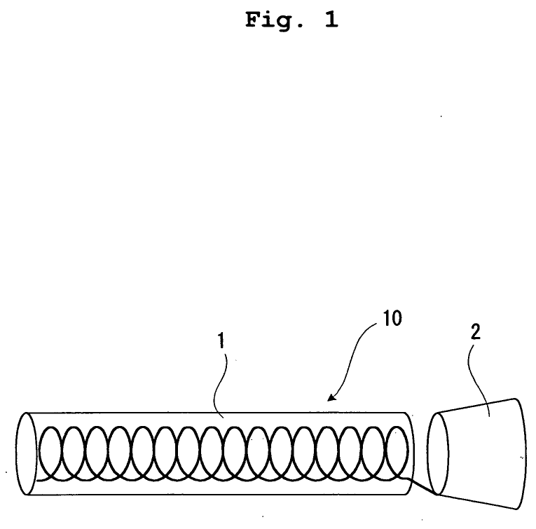

As shown as an example in FIG. 1, the waste water inspecting agent 10 of the present invention is used as additive to a specimen and has a rod-shaped body 1 and a capturing structured element 2 which specifically captures, by bonding to the rod-shaped body, an object to be captured contained in the specimen solution. Incidentally, in FIG. 1, the capturing structured element 2 is bonded to one end of the rod-shaped body 1. However, it may be also bonded to the circumferential side of the rod-shaped body 1 and, in that case, it is also possible to have a plural capturing structured element bonded to the circumferential side of the rod-shaped body.

<Rod-Shaped Body>

The rod-shaped body is not particularly limited provided that it is rod-shaped, and may be appropriately selected in accordance with the object. The rod-shaped body may be either a rod-shaped inorganic substance or rod-shaped organic substance, but a rod-shaped organic substance is preferable.

Examples of rod-shaped organic substances are biopolymers, polysaccharides, and the like.

Suitable examples of biopolymers are fibrous proteins, α-helix polypeptides, nucleic acids (DNA, RNA), and the like. Examples of fibrous proteins are fibrous proteins having α-helix structures such as α-keratin, myosin, epidermin, fibrinogen, tropomyosin, silk fibroin, and the like. Suitable examples of polysaccharides are amylose and the like.

Among rod-shaped organic substances, spiral organic molecules whose molecules have a spiral structure are preferable from the standpoints of stable maintenance of the rod shape and internal intercalatability of other substances in accordance with an object. Among the aforementioned substances, those with spiral organic molecules include α-helix polypeptides, DNA, amylose, and the like.

{α-Helix Polypeptides}

α-helix polypeptides are referred to as one of the secondary structures of polypeptides. The polypeptide rotates one time (forms one spiral) for each amino acid 3.6 residue, and a hydrogen bond, which is substantially parallel to the axis of the helix, is formed between a carbonyl group (—CO—) and an imide group (—NH—) of each fourth amino acid, and this structure is repeated in units of seven amino acids. In this way, the α-helix polypeptide has a structure which is stable energy-wise.

The direction of the spiral of the α-helix polypeptide is not particularly limited, and may be either wound right or wound left. Note that, in nature, only structures whose direction of spiral is wound right exist from the standpoint of stability.

The amino acids which form the α-helix polypeptide are not particularly limited provided that an α-helix structure can be formed, and can be appropriately selected in accordance with the object. However, amino acids which facilitate formation of the α-helix structure are preferable. Suitable examples of such amino acids are aspartic acid (Asp), glutamic acid (Glu), arginine (Arg), lysine (Lys), histidine (His), asparagine (Asn), glutamine (Gln), serine (Ser), threonine (Thr), alanine (Ala), valine (Val), leucine (Leu), isoleucine (Ile), cysteine (Cys), methionine (Met), tyrosine (Tyr), phenylalanine (Phe), tryptophan (Trp), and the like. A single one of these amino acids may be used alone, or two or more may be used in combination.

By appropriately selecting the amino acid, the property of the α-helix polypeptide can be changed to any of hydrophilic, hydrophobic, and amphiphilic. In the case in which the α-helix polypeptide is to be made to be hydrophilic, suitable examples of the amino acid are serine (Ser), threonine (Thr), aspartic acid (Asp), glutamic acid (Glu), arginine (Arg), lysine (Lys), asparagine (Asn), glutamine (Gln), and the like. In the case in which the α-helix polypeptide is to be made to be hydrophobic, suitable examples of the amino acid are phenylalanine (Phe), tryptophan (Trp), isoleucine (Ile), tyrosine (Tyr), methionine (Met), leucine (Leu), valine (Val), and the like.

In the α-helix polypeptide, the carboxyl group, which does not form a peptide bond and which is in the amino acid which forms the α-helix, can be made to be hydrophobic by esterification. On the other hand, an esterified carboxyl group can be made to be hydrophilic by hydrolysis.

The amino acid may be any of a L-amino acid, a D-amino acid, a derivative in which the side chain portion of a L-amino acid or a D-amino acid is modified, and the like.

The number of bonds (the degree of polymerization) of the amino acid in the α-helix polypeptide is not particularly limited and may be appropriately selected in accordance with the object. However, 10 to 5000 is preferable.

If the number of bonds (the degree of polymerization) is less than 10, it may not be possible for the polyamino acid to form a stable α-helix. If the number of bonds (the degree of polymerization) exceeds 5000, vertical orientation may be difficult to achieve.

Suitable specific examples of the α-helix polypeptide are polyglutamic acid derivatives such as poly(γ-methyl L-glutamate), poly(γ-ethyl L-glutamate), poly(γ-benzyl glutamate), poly(n-hexyl glutamate), and the like; polyaspartic acid derivatives such as poly(β-benzyl L-aspartate) and the like; polypeptides such as poly(L-leucine), poly(L-alanine), poly(L-methionine), poly(L-phenylanine), poly(L-lysine)-poly(γ-methyl L-glutamate), and the like.

The α-helix polypeptide may be a commercially available α-helix polypeptide, or may be appropriately synthesized or prepared in accordance with methods disclosed in known publications and the like.

As one example of synthesizing the α-helix polypeptide, the synthesis of block copolypeptide [poly(L-lysine)₂₅-poly(γ-methyl L-glutamate)₆₀]PLLZ₂₅-PMLG₆₀ is as follows. As is shown by the following formula, block copolypeptide [poly(L-lysine)₂₅-poly(γ-methyl L-glutamate)₆₀]PLLZ₂₅-PMLG₆₀ can be synthesized by polymerizing N^{ε}-carbobenzoxy L-lysine N^{α}-carboxy acid anhydride (LLZ-NCA) by using n-hexylamine as an initiator, and then polymerizing γ-methyl L-glutamate N-carboxy acid anhydride (MLG-NCA).

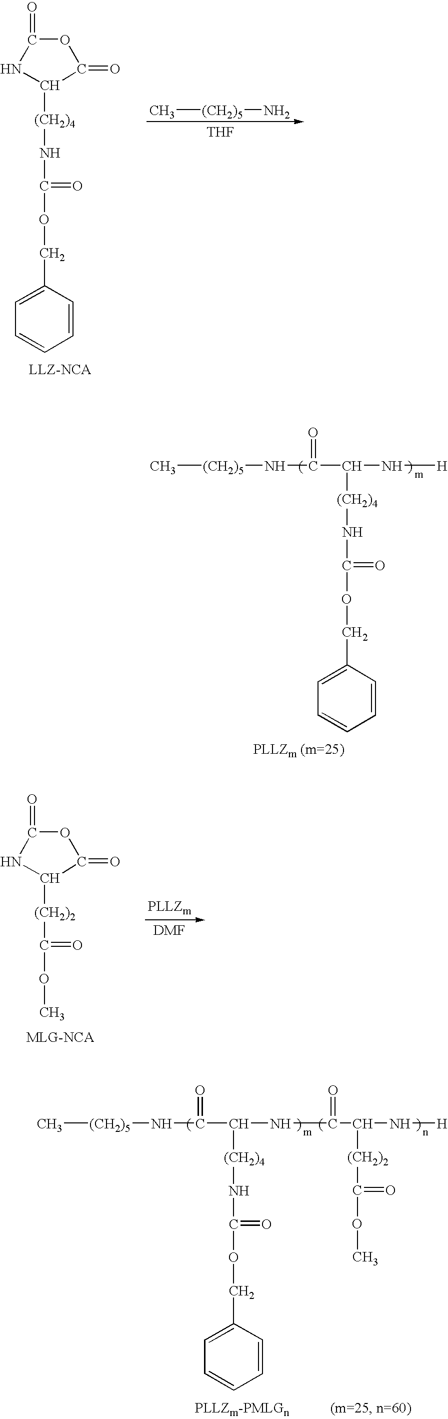

Synthesis of the α-helix polypeptide is not limited to the above-described method, and the α-helix polypeptide can be synthesized by a genetic engineering method. Specifically, the α-helix polypeptide can be manufactured by transforming a host cell by a expression vector in which is integrated a DNA which encodes the target polypeptide, and culturing the transformant, and the like.

Examples of the expression vector include a plasmid vector, a phage vector, a plasmid and phage chimeric vector, and the like.

Examples of the host cell include prokaryotic microorganisms such as E. coli, Bacillus subtilis, and the like; eukaryotic microorganisms such as yeast and the like; zooblasts, and the like.

The α-helix polypeptide may be prepared by removing the α-helix structural portion from a natural fibrous protein such as α-keratin, myosin, epidermin, fibrinogen, tropomyosin, silk fibroin, and the like.

{DNA}

The DNA may be a single-stranded DNA. However, the DNA is preferably a double-stranded DNA from the standpoints that the rod-shape can be stably maintained, other substances can be intercalated into the interior, and the like.

A double-stranded DNA has a double helix structure in which two polynucleotide chains, which are in the form of right-wound spirals, are formed so as to be positioned around a single central axis in a state in which they extend in respectively opposite directions.

The polynucleotide chains are formed by four types of nucleic acid bases which are adenine (A), thiamine (T), guanine (G), and cytosine (C). The nucleic acid bases in the polynucleotide chain exist in the form of projecting inwardly within a plane which is orthogonal to the central axis, and form so-called Watson-Crick base pairs. Thiamine specifically hydrogen bonds with adenine, and cytosine specifically hydrogen bonds with guanine. As a result, in a double-stranded DNA, the two polypeptide chains are bonded complementarily.

The DNA can be prepared by known method such as PCR (Polymerase Chain Reaction), LCR (Ligase Chain Reaction), 3SR (Self-Sustained Sequence Replication), SDA (Strand Displacement Amplification), and the like. Among these, the PCR method is preferable.

Further, the DNA can be prepared by being directly removed enzymatically from a natural gene by a restriction enzyme. Or, the DNA can be prepared by a genetic cloning method, or by a chemical synthesis method.

In the case of a genetic cloning method, a large amount of the DNA can be prepared by, for example, integrating a structure, in which a normal nucleic acid has been amplified, into a vector which is selected from plasmid vectors, phage vectors, plasmid and phage chimeric vectors, and the like, and then introducing the vector into an arbitrary host in which propagation is possible and which is selected from prokaryotic microorganisms such as E. coli, Bacillus subtilis, and the like; eukaryotic microorganisms such as yeast and the like; zooblasts, and the like.

Examples of chemical synthesis methods include liquid phase methods or solid phase synthesis methods using an insoluble carrier, such as a tolyester method, a phosphorous acid method, and the like. In the case of a chemical synthesis method, the double-stranded DNA can be prepared by using a known automatic synthesizing device and the like to prepare a large amount of single-stranded DNA, and thereafter, carrying out annealing.

{Amylose}

Amylose is a polysaccharide having a spiral structure in which D-glucose, which forms starch which is a homopolysaccharide of higher plants for storage, is joined in a straight chain by α-1,4 bonds.

The molecular weight of the amylose is preferably around several thousand to 150,000 in number average molecular weight.

The amylose may be a commercially available amylose, or may be appropriately prepared in accordance with known methods.

Amylopectin may be contained in a portion of the amylose.

The length of the rod-shaped body is not particularly limited, and may be appropriately selected in accordance with the object. However, from the standpoint of causing reflection of the incident light as colored interference light which will be described later, a length of 810 nm or less is preferable, and 10 nm to 810 nm is more preferable.

The diameter of the rod-shaped body is not particularly limited, and is about 0.8 to 2.0 nm in the case of the α-helix polypeptide.

The entire rod-shaped body may be hydrophobic or hydrophilic. Or, the rod-shaped body may be amphiphilic such that a portion thereof is hydrophobic or hydrophilic, and the other portion thereof exhibits the opposite property of the one portion. In the case of an amphiphilic rod-shaped body, the numbers of the lipophilic (hydrophobic) portions and hydrophilic portions are not particularly limited, and may be appropriately selected in accordance with the object. Further, in this case, the portions which are lipophilic (hydrophobic) and the portions which are hydrophilic may be positioned alternately, or either type of portion may be positioned only at one end portion of the rod-shaped body.

In the case of the amphiphilic rod-shaped body, there is no particular limitation for the numbers of the moiety showing hydrophobicity and the moiety showing hydrophilicity but that may be appropriately selected according to the object. In that case, the moiety showing hydrophobicity and the moiety showing hydrophilicity may be alternately positioned. Any of the moieties may be positioned only at one end of the rod-shaped body.

{Capturing Structured Element}

The capturing structured element is not particularly limited provided that it is able to capture the object to be captured (or an object to be captured) and may be suitably selected according to an object.

Examples of capturing modes include, but are not limited to, physical adsorption, chemical adsorption, and the like. These modes allows formation of bonds by, for example, by hydrogen bonding, intermolecular forced (van der Waals force), coordinate bonding, ionic bonding, covalent bonding, and the like.

Particular examples of the capturing structured element preferably include, host components involved in clatharate compound (hereinafter, interchangeably referred to as “host”), antibody, nucleic acid, hormone receptor, lectin, and physiologically active agent receptor. Among all, nucleic acid is preferred in view of easy formation of any alignment and more preferably, single-stranded DNA or single-stranded RNA.

With regard to an object to be captured of such a capturing structured element, which may be a guest (component to be included) in the case of clatharate compound, an antigen in the case of antibody, a nucleic acid, a tubulin, a chitin and the like in the case of nucleic acid, a hormone in the case of hormone, sugar and the like in the case of lectin, and a physiologically active substance in the case of physiologically active agent receptor.

{Clatharate Compound}

The clatharate compound is not particularly limited provided that it posses molecular recognizing ability (host-guest binding ability) and may be appropriately selected according to an object. Preferable examples of such clatharate compound include the ones having tubular (one-dimensional) hollow, or layer-shaped (two-dimensional) hollow, or cage-shaped (three-dimensional) hollow, and the like.

Examples of the clatharate compound having the tubular (one-dimensional) hollow are, urea, thiourea, deoxycholic acid, dinitrodiphenyl, dioxytriphenylmethane, triphenylmethane, methylnaphthalene, spirochroman, PHTP (perhydrotriphenylene), cellulose, amylose, cyclodextrin (where the hollow is cage-shaped in a solution), phenylboric acid, and the like.

Examples of an object to be captured (the guest) by the urea, may be n-paraffin derivatives, and the like.

Examples of an object to be captured (the guest) by the thiourea, may be branched or cyclic hydrocarbons and the like.

Examples of an object to be captured (the guest) by the deoxycholic acid, may be paraffins, fatty adds, aromatic compounds, and the like

Examples of an object to be captured (the guest) by the dinitrodiphenyl, may be diphenyl derivatives, and the like.

Examples of an object to be captured (the guest) by the dioxytriphenylmethane, may be paraffins, n-alkenes, squalene, and the like.

Examples of an object to be captured (the guest) by the triphenylmethane, may be paraffins, and the like.

Examples of an object to be captured (the guest) by the methylnaphthalene, may be C₁₆ or less n-paraffins, branched paraffins, and the like.

Examples of an object to be captured (the guest) by the spirochroman, may be paraffins, and the like.

Examples of an object to be captured (the guest) by the PHTP (perhydrotriphenylene), may be chloroform, benzene, various high-molecular substances, and the like.

Examples of an object to be captured (the guest) by the cellulose, may be H₂O² paraffins, CCl₄, dyes, iodine, and the like.

Examples of an object to be captured (the guest) by the amylose, may be fatty acids, iodine, and the like.

The cyclodextrin is a cyclic dextrin which is formed by degradation of starch using amylase and three types are presently known. Namely, α-cyclodextrin, β-cyclodextrin and γ-cyclodextrin. In the present invention, the cyclodextrin includes cyclodextrin derivatives where a part of hydroxyl groups thereof are substituted with other functional group such as, for example, alkyl group, allyl group, alkoxy group, amide group, sulfonic acid group, and the like.

Examples of an object to be captured (the guest) by the cyclodextrin, may be phenyl derivatives such as thymol, eugenol, resorcinol, ethylene glycol monophenyl ether, 2-hydroxy-4-methoxybenzophenone, and the like, benzoic acid derivatives and esters thereof such as salicylic acid, methyl p-hydroxybenzoate, ethyl p-hydroxybenzoate, and the like, steroids such as cholesterol, and the like, vitamins such as ascorbic acid, retinol, tocopherol, and the like, hydrocarbons such as limonene, and the like, allyl isothiocyanate, sorbic acid, iodine molecule, Methyl Orange, Congo Red, potassium 2-p-toluidinylnaphthalene-6-sulfonate (TNS), and the like.

Examples of an object to be captured (the guest) by the phenylboric acid, may be glucose, and the like.

Examples of a layered (two-dimensional) clatharate compound, may be clay mineral, graphite, smectite, montmorillonite, zeolite, and the like.

Examples of an object to be captured (the guest) by the clay mineral, may be hydrophilic substances, polar compounds, and the like.

Examples of an object to be captured (the guest) by the graphite, may be O, HSO₄⁻, halogens, halides, alkaline metals, and the like.

Examples of an object to be captured (the guest) by the montmorillonite, may be brucine, codeine, o-phenylenediamine, benzidine, piperidine, adenine, guianine and liposide thereof, and the like.

Examples of an object to be captured (the guest) by the zeolite, may be H₂O, and the like.

With regard to the cage-shaped (three-dimensional) clatharate compound, examples include hydroquinone, gaseous hydrate, tri-o-thymotide, oxyflavan, dicyanoammine nickel, cryptand, calixarene, crown compound, and the like.

Examples of an object to be captured (the guest) by the hydroquinone, may be HCl, SO₂, acetylene, rare gas elements, and the like.

Examples of an object to be captured (the guest) by the gaseous hydrate, may be halogens, rare gas elements, lower hydrocarbons, and the like.

Examples of an object to be captured (the guest) by the tri-o-thymotide, may be cyclohexane, benzene, chloroform, and the like.

Examples of an object to be captured (the guest) by the oxyflavan, may be organic bases, and the like.

Examples of an object to be captured (the guest) by the dicyanoammine nickel, may be benzene, phenol, and the like.

Examples of an object to be captured (the guest) by the cryptand, may be NH₄⁺, various metal ions, and the like.

The calixarene is a cyclic oligomer where a phenol unit synthesized from phenol and formaldehyde under a suitable condition is bonded to a methylene unit and its 4- to 8-nuclear substances are known. Among them, examples of an object to be captured (the guest) by the p-tert-butylcarixarene (n=4) may include, chloroform, benzene, toluene, and the like, examples of an object to be captured (the guest) by the p-tert-butylcarixarene (n=5) may include, isopropyl alcohol, acetone, and the like, examples of an object to be captured (the guest) by the p-tert-butylcarixarene (n=6) may include, isopropyl alcohol, acetone, and the like, chloroform, methanol, and the like, and examples of an object to be captured (the guest) by the p-tert-butylcarixarene (n=7) may include, chloroform, and the like.

The crown compound includes a macro cyclic compound having not only a crown ether having oxygen as an electron-donating donor atom but also donor atom such as nitrogen, sulfur, and the like as an analog thereof as constituting elements for a ring structure, and also includes a multicyclic crown compound comprising two or more rings represented by cryptand for example, cyclohexyl-12-crown-4, dibenzo-14-crown-4, tert-butylbenzo-15-crown-5, dibenzo-18-crown-6, dicyclohexyl-18-crown-6, 18-crown-6, tribenzo-18-crown-6, tetrabenzo-24-crown-8, dibenzo-26-crown-6, and the like.

Examples of an object to be captured (the guest) by the crown compound, may be various metal ions such as alkaline metals (e.g., Li, Na and K) and alkaline earth metals (e.g., Mg and Ca), NH₄⁺, alkylammonium ion, guanidium ion, aromatic diazonium ion, and the like and the crown compound forms a complex therewith. Examples of an object to be captured (the guest) by the crown compound, may further include polar organic compounds having C—H (acetonitrile, malononitrile, adiponitrile, and the like), N—H (aniline, aminobenzoic acid, amide, sulfamide derivative, and the like) and O—H (phenol, acetic add derivative, and the like), unit where acidity is relatively high and the crown compound forms a complex therewith.

The size (or the diameter) of the hollow of the clatharate compound is not particularly limited and may be suitably selected according to an object. However, from a standpoint of achieving stable molecular recognizing ability (host-guest binding ability), 0.1 nm to 2.0 nm in diameter is preferred.

A mixing rate (molar ratio) of the clatharate compound (host) to the guest cannot be determined at a fixed rate, and may differ according to the type of the clatharate compound and the type of the guest. However usually the rate (clatharate compound):(guest component) is from 1:0.1 to 1:10 and, preferably, from 1:0.3 to 1:3.

{Antibody}

The antibody is not particularly limited provided that it causes an antigen-antibody reaction specifically with the target antigen (object to be captured). As such, it may be either a polyclonal antibody or a monoclonal antibody and it also may be Fab′, Fab, F(ab′)₂, and the like of IgG, IgM, IgE and IgG.

There is no particular limitation for the target antigen but it may be appropriately selected depending on the object. Examples include plasma protein, tumor marker, apoprotein, virus, autoantibody, coagulation/fibrinolysis factor, hormone, blood drugs, HLA antigen, and the like.

{Protein Having Affinity to Heavy Metals}

The protein of a low molecular weight (about 6000-13,000) having a high affinity to many heavy metals, particularly to zinc, cadmium, copper, mercury, and the like, existing in liver, kidney and other tissues of animals and being also found in microbes recently. In addition, such a protein contains certain amount of cysteine, shows an amino acid distribution containing almost no aromatic residue and is an important substance having a detoxicating function for cadmium, mercury, and the like in vivo and participating in storage of essential minor metal for living body such as zinc and copper and in distribution thereof in vivo as well.

{Object to be Captured}

The object to be captured is preferably at least one material selected from heavy metals, toxic organic compounds, agricultural chemicals, endocrine disruptors in the environment and genetic recombinant cells. It is not necessary that the object to be captured is not the final target substance for the detection but may be a substance which coexist with the final target substance for the detection.

For the above-mentioned heavy metals, examples such as alkyl mercury compound (R—Hg), mercury or its compound (Hg), cadmium or its compound (Cd), lead or its compound (Pb), hexavalent chromium (Cr⁶⁺), copper or its compound (Cu), zinc or its compound (Zn), cyan, hexavalent chromium, arsenic, selenium, manganese, nickel, iron, zinc, selenium, tin, and the like may be used.

For the toxic organic compounds, examples such as cyan compound, phenols, dichloromethane, ammonia, carbon tetrachloride, 1,2-chloroethane, 1,1-dichloroethylene, cis-1,2-dichloroethylene, 1,1,1-trichloroethane, 1,1,2-trichloroethane, trichloroethylene, tetrachloroethylene, benzene, 1,3-dichlorobenzene, dioxin, PCB, DDT, DES, and the like may be used.

For the agricultural chemicals, there may be examples such as organ phosphorus, 1,3-dichloropropene, thiraum, simazine, thiobencarb, and the like may be used.

For the endocrine disruptors in the environment, examples include bisphenol A, nonylphenol, phthalates, organotin compounds, DDT, PCB, dioxins, and the like.

For the genetic recombinant cells, examples include corn, rice plant, tomato, and the like.

The waste water inspecting agent of the present invention is obtained by bonding the rod-shaped body and the capturing structured element having an ability to recognize the object to be captured.

The bonding method may be appropriately selected according to the capturing structured element and the rod-shaped body. Known methods include a method where a covalent bond such as ester bond or amide bond is utilized, a method where protein is labeled with avidin and is bonded to a biotinated capturing structured element, a method where protein is labeled with streptoavidin and is bonded to a biotinated capturing structured element, and the like.

Examples of the covalent bond method includes, peptide method, diazo method, alkylation method, cyan bromide activation method, bonding by a cross-linking reagent, immobilization utilizing Ugi reaction, immobilization utilizing a thiol-disulfide exchange reaction, Schiff base formation method, chelate bonding method, tosyl chloride method, biochemically specific bonding method, and the like. For more stable bonding such as covalent bond, preferably a reaction of thiol group with maleimide group, reaction of pyridyl disulfide group with thiol group, reaction of pyridyl disulfide group with thiol group, reaction of amino group with aldehyde group, and the like may be utilized and known methods, methods simply carried out by those skilled in the art, and modified methods thereof may be utilized. Among them, a chemically bonding agent and a cross-linking agent may form a more stable bonding.

With regard to such chemically bonding agent and cross-linking agent, there may be exemplified carbodiimide, isocyanate, diazo compound, benzoquinone, aldehyde, periodic acid, maleimide compound, pyridyl disulfide compound, and the like. With regard to the preferred reagent, there may be exemplified glutaraldehyde, hexamethylene diisocyanate, hexamethylene diisothiocyanate, N,N′-polymethylenebisiodoacetamide, N,N′-ethylenebismaleiimide, ethylene glycol bissuccinimidyl succinate, bisdiazobenzidine, 1-ethyl-3-(3-dimethylaminopropyl)carbodiimide, succinimidyl 3-(2-pyridylthio)propionate (SPDP), N-succinmidyl (N-maleimidomethyl)cyclohexane-1-carboxylate (SMCC), N-sulfosuccinimidyl 4-(N-maleimidomethyl)cyclohexane-1-carboxylate, N-succinmidyl(4-iodoacetyl)aminobenzoate, N-succinimidyl 4-(1-maleimidophenyl)butyrate, iminothiolane, S-acetylmercaptosuccinic acid anhydride, methyl-3-(4′-dithiopyridyl)propionimidate, methyl-4-mercaptobutyryl imidate, methyl-3-mercaptopropionimidate, N-succinimidyl-S-acetyl mercaptoacetate, and the like.

{Waste Water Inspecting Agent}

In the waste water inspecting agent, when the object to be captured is captured by the capturing structured element, physical properties of the waste water inspecting agent such as refractive index and transmittance of light, mass, viscoelasticity, and the like change and, therefore, when the change is detected, it may be utilized for the detection of the captured object. The above method for the detection may be appropriately selected according to the object and, for example, various methods such as that color change is observed by naked eye, that wavelength change is detected by spectrophotometer, that oscillation of frequency of quartz oscillator, surface acoustic wave (SAW) element and the like is detected by a frequency counter, and the like may be carried out.

The waste water inspecting agent may be used alone, and in that case, when it is used by aligning in single or plural layer(s) on the surface of a solvent containing the subject to be captured or at the boundary between the solvent and a liquid immisible to the solvent is preferred, since changes in wavelength may easily be detected.

It is also possible to form in a film like manner such as monomolecular film or bimolecular film on a substrate which is vertically aligned by, for example, a Langmuir-Brodgett (LB) technique.

With regard to the waste water inspecting agent of the present invention, the one which is able to reflect the incident light as colored interference light is preferred from a viewpoint of recognition and discrimination.

The reflection of the incident light as colored interference light is a color formation on the basis of a multi-layer thin film interference theory which is a basic principle for color formation of the scaly powder of the wings of a Morpho butterfly and is a color formation on the film as a result of reflection of light of specific wavelength corresponding to the thickness of the film and the refractivity thereof when stimulation from outside such as electric field, magnetic field, heat, light (for example, natural light, infrared light and ultraviolet light), and the like is applied to the film. The color tone may be freely controlled like the surface skin of a chameleon by the stimulation from outside.

Principle of light reflection of an incident light as colored interference light will be described hereinafter.

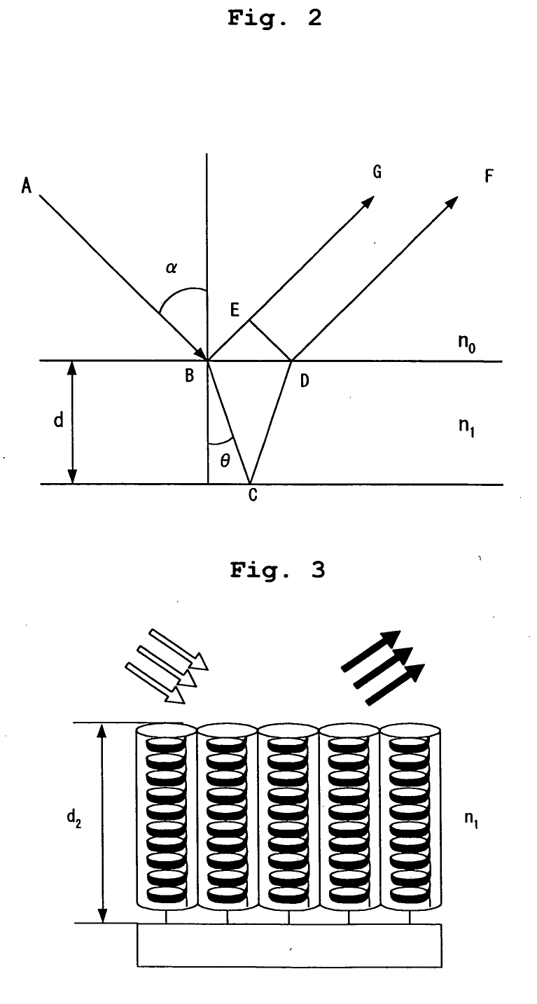

As shown in FIG. 2 and FIG. 3, when light is irradiated on the film of the rod-shaped body, wavelength (λ) of the interference light by the film is emphasized under the condition as shown in the following (1) and enfeebled under the condition as shown in the following (2).

$$\begin{matrix}λ = \frac{2 tl}{m} \sqrt{{n}^{2} - {sin}^{2} α} & ( 1 ) \\ λ = \frac{4 tl}{2 m - 1} \sqrt{{n}^{2} - {sin}^{2} α} & ( 2 )\end{matrix}$$

In the formulae (1) and (2), λ means wavelength (nm) of the interference light, a means angle of incidence (degree) of the light to the film, t means thickness (nm) of a single film, 1 means number of layers of the film, n means a refractive index of the film and m means an integer of 1 or more.

The light reflection of the incident light as colored interference light may be obtained by aligning the waste water inspecting agent into a film-like shape.

Thickness of the single film is preferably 810 nm or less and, more preferably, it is from 10 nm to 810 nm.

When the thickness is appropriately changed, color (wavelength) of the light reflection of the incident light as interference light may be changed.

The film may be either a monomolecular film or a two layered monomolecular films.

The monomolecular film or the layered films comprising the same may be formed by, for example, a Langmuir-Brodgett method (LB method) and, in that case, a known LB film forming apparatus (such as NL-LB 400 NK-MWC manufactured by Nippon Laser & Electronics Laboratories) may be used.

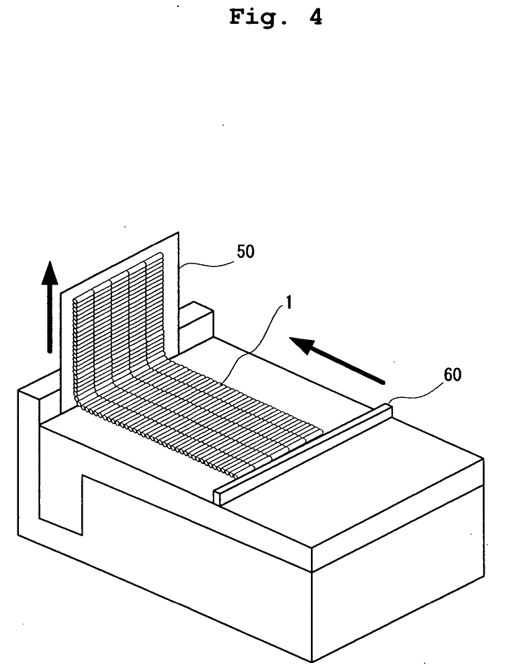

Formation of the monomolecular film may be carried out, for example, in such a state that the rod-shaped body which is lipophilic (hydrophobic) or amphiphilic is floated on water surface (on an aqueous phase) or in such a state that the rod-shaped body which is lipophilic (hydrophobic) or amphiphilic is floated on oil surface (on an oil phase) or, in other words, the rod-shaped body 1 is aligned as shown in FIG. 4 so as to form on a substrate 50 using an pushing material 60. When such an operation is repeatedly carried out, the layered films where the monomolecular films are layered in any number may be formed on the substrate 50. Incidentally, it is preferred that the monomolecular film or the layered film is fixed on the substrate 50 since the reflection of the incident light as colored interference light by the monomolecular film or layered films is expressed in a stable manner.

In that case, there is no particular limitation for the substrate 50 and, according to the object, its material, shape, size, and the like may be appropriately selected although it is preferred that its surface is appropriately subjected to a surface treatment previously with an object that the rod-shaped body 1 is easily adhered or bonded thereto. When the rod-shaped body 1 (such as α-helix polypeptide) is hydrophilic for example, it is preferred that a surface treatment such as hydrophilizing treatment using octadecyl trimethylsiloxane and the like is previously carried out.

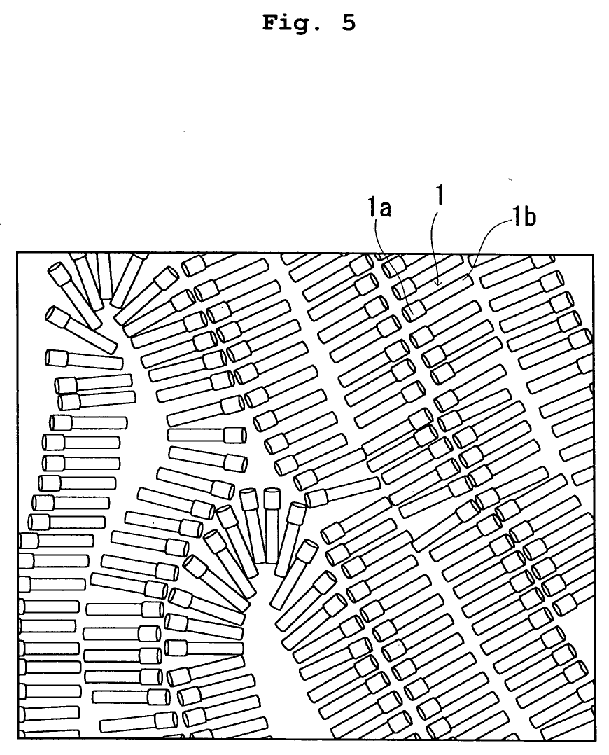

With regard to the state where the rod-shaped body is floated on an oil phase or an aqueous phase in the formation of the monomolecular film of the amphiphilic rod-shaped body, the lipophilic areas (hydrophobic areas) 1a of the rod-shaped body 1 are aligned in an adjacent state to each other on the aqueous phase or oil phase while the hydrophilic areas lb are aligned in an adjacent state each other as shown in FIG. 5.

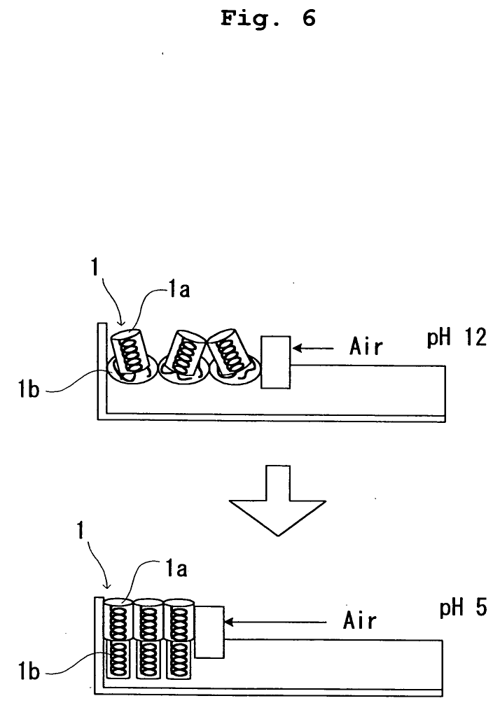

The above is an example of a layered membrane or a layered films comprising the same where the rod-shaped body is aligned in the plane direction of the monomolecular film (in a horizontal state) while a monomolecular film where the rod-shaped body is aligned in the thickness direction of the monomolecular film (in a vertical state) may be manufactured, for example, as follows. First, as shown in FIG. 6, water (aqueous phase) is made alkaline of around pH 12 under such a state that the amphiphilic rod-shaped body 1 (α-helix polypeptide) is floated on the water surface (aqueous phase) (i.e., in a horizontal state). As a result, in the hydrophilic area lb in the rod-shaped body 1 (α-helix polypeptide), the α-helix structure thereof is disentangled to give a random structure. At that time, the lipophilic area (hydrophobic area) 1a of the rod-shaped body 1 (α-helix polypeptide) maintains its α-helix structure. Then, the pH of the water (aqueous phase) is made acidic to about 5 thereby the hydrophilic area 1b in the rod-shaped body 1 (α-helix polypeptide) forms an α-helix structure again. When the pushing material attached to the rod-shaped body 1 (α-helix polypeptide) is pushed by the pressure of air from its side to the rod-shaped body 1 (α-helix polypeptide), the rod-shaped body 1 maintains vertical against water (aqueous phase) while its hydrophilic area 1b forms an α-helix structure in the direction substantially orthogonal to the water surface in the aqueous phase. When the aligned rod-shaped body 1 (α-helix polypeptide) is pushed out onto the substrate 50 using a pushing material 60 as mentioned above by referring to FIG. 4, it is possible to form a monomolecular film on the substrate 50. When such operation is repeatedly carried out, the layered films having prescribed number of monomolecular film may be formed on the substrate 50.

Example of the waste water inspecting agent having a single layer or laminated layers which may reflect the incident light as colored interference light could be an amphiphilic waste water inspecting agent, and preferably the rod-shaped body is α-helix polypeptide.

Waste water inspecting agent of the present invention may be a inspecting agent which precipitates or form gels when object to be captured is captured.

The waste water inspecting agent of the preset invention is not particularly limited. The waste water inspecting agent is added to the waste water discharged from factories, and the like and changes in color tone or wavelength of the light reflection of the incident light as colored interference light by capturing the object to be captured by the waste water inspecting agent are measured whereby the presence of the object to be captured in the waste water may be confirmed. It is preferred that the waste water inspecting agent is added in a form of a state of emulsion to the waste water or the like to measure the changes in color tone or wavelength.

To be specific, when a protein (metallothioneine or thioneine-like protein) having an affinity to heavy metals is used as the object to be captured and a waste water inspecting agent where the protein is bonded to a rod-shaped body is used, the heavy metals (zinc, cadmium copper, mercury, and the like) in the waste water may be detected.

When a waste water inspecting agent in which a crown ether compound is used as a capturing structure material and bonded to a rod-shaped body is used, various metal ions in the waste water such as alkaline metals (Li, Na, K, and the like), alkaline earth metals(Mg, Ca, and the like), and the like may be detected.

When a waste water inspecting agent in which cyclodextrin is used as a capturing structure material and is bonded to the rod-shaped body is used, presence of the toxic organic compounds, and the like in the waste water may be confirmed.

When a waste water inspecting agent in which an antibody to endocrine disruptors in the environment such as bisphenol A, nonylphenyl, phthalate, and the like is used as a capturing structure material and is bonded to the rod-shaped body is used, presence of the endocrine disruptors in the environment in the waste water may be confirmed.

<Waste Water Inspecting Apparatus>

The first embodiment of the waste water inspecting apparatus of the present invention is equipped with a waste water inspecting agent having a rod-shaped body and a capturing structure material which captures the object to be captured contained in the waste water to be inspected which is bonded to the rod-shaped body and reflecting the incident light as colored interference light by aligning in a film-like shape; an adding means where the waste water inspecting agent is contacted to a sample; and a colored wavelength measuring means in which changes in wavelength by light reflection of the colored interference light of the film like waste water inspecting agent which is bonded to the object to be captured are measured.

For the liquid to be inspected, there is no particular limitation as long as it contains an object to be captured which is an object for the inspection and there may be examples such as water in the rivers and streams, waste water from factories, and the like.

With regard to the adding means, there is no particular limitation so far as it is a means for adding a predetermined amount of the waste water inspecting agent is added to the sample to be examined or it is a means for adding a predetermined amount of the sample to be examined to the waste water inspecting agent. It is preferred however that the amount of the waste water inspecting agent is made to such an extent that the light reflection of the incident light as colored interference light may be easily detected by aligning in a film-like shape.

One of the preferred aspects of the waste water inspecting apparatus is an aspect in which the waste water inspecting agent is amphiphilic and the adding means is a means for adding the waste water inspecting agent and the oil phase thereof into an aqueous sample and for bringing the waste water inspecting agent to contact the sample.

In that case, the waste water inspecting agent is amphiphilic in which the waste water inspecting agent is aligned vertically to comprise a film-like shape at the interface between the oil phase and the aqueous phase and, therefore, it is preferred because changes in wavelength caused by the light reflection of the incident light as colored interference light are easily measured.

The waste water inspecting apparatus in accordance with the second aspect of the present invention is provided with a biosensor where the waste water inspecting agent of the present invention is adhered and bonded in a film-like shape to a quartz oscillator or a surface acoustic wave (SAW) element, an oscillation circuit where changes in mass or changes in viscoelasticity when the object to be captured is captured by the biosensor are oscillated as a frequency and a frequency counter where the frequency of the oscillation oscillated from the oscillation circuit is measured.

In that case, it is preferred that the waste water inspecting agent is adhered and bonded in a monomolecular film-like shape to the quartz oscillator or to the surface acoustic wave (SAW) element or is adhered and bonded in a bimolecular film-like shape thereto. With regard to the frequency counter, there is no particular limitation so far as it is able to precisely measure the frequency from the quartz oscillator or the surface acoustic wave (SAW) element.

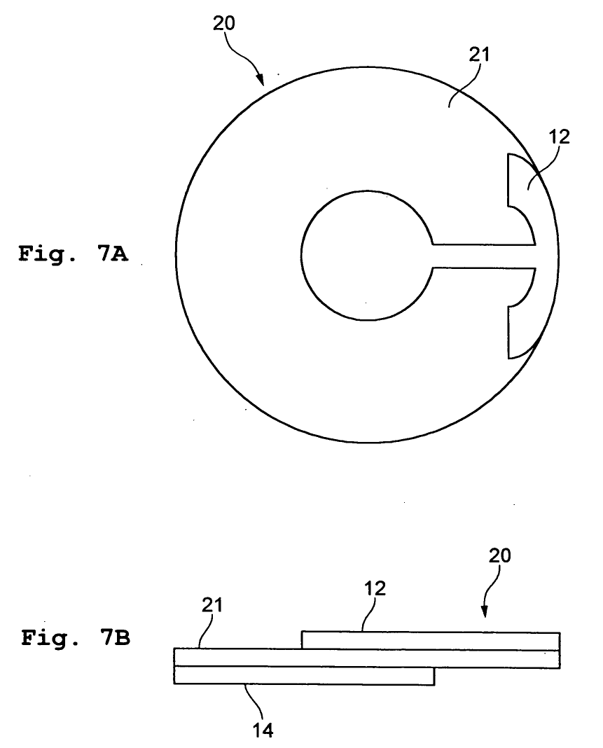

In the quartz oscillator, metal electrodes are vapor deposited on the surface and the back of a thin quartz plate. An example of the quartz oscillator 20 is shown in FIGS. 7A and 7B. FIG. 7A is a plane view while FIG. 7B is a front view. An electrode 12 is vapor deposited on the surface of the quartz plate 21 while another electrode 14 is vapor deposited on the back thereof. The electrodes extend to the left side from the electrodes 12, 14 and the left ends thereof are connected to clip-type lead wires (not shown) followed by connecting to an alternating current source (not shown). When alternating current is applied between the electrodes 12, 14, there is generated oscillation of a predetermined period in the quartz plate 21 due to a back piezoelectric effect.

On the surface of the quartz oscillator 20, there is adhered and bonded a waste water inspecting agent film (not shown). The capturing bonding material of this waste water inspecting agent film captures the object to be captured and mass of the surface of the quartz oscillator 20 changes corresponding to the mass of the captured disease marker whereby the resonance frequency changes.

Between the changes in the resonance frequency and changes in the mass of the waste water inspecting agent film coated on the surface of the quartz oscillator 20 which oscillates in parallel to the plane vertical to the thickness direction, there is a relation as shown in the following formula (3) whereby changes in the mass may be detected from changes in the resonance frequency. For example, in the case of an oscillator of resonance frequency of 9 MHz (area: about 0.5 cm²), a reduction in frequency of 400 Hz is resulted by an increase in mass of 1 μg.
ΔF=−2.3×10⁶(F²×ΔW/A) (3)

In the formula, F means resonance frequency (MHz) of the quartz oscillator, ΔF means changes (Hz) in the resonance frequency by changes in mass, ΔW means changes in mass (g) of the film and A means a surface area (cm²) of the film.

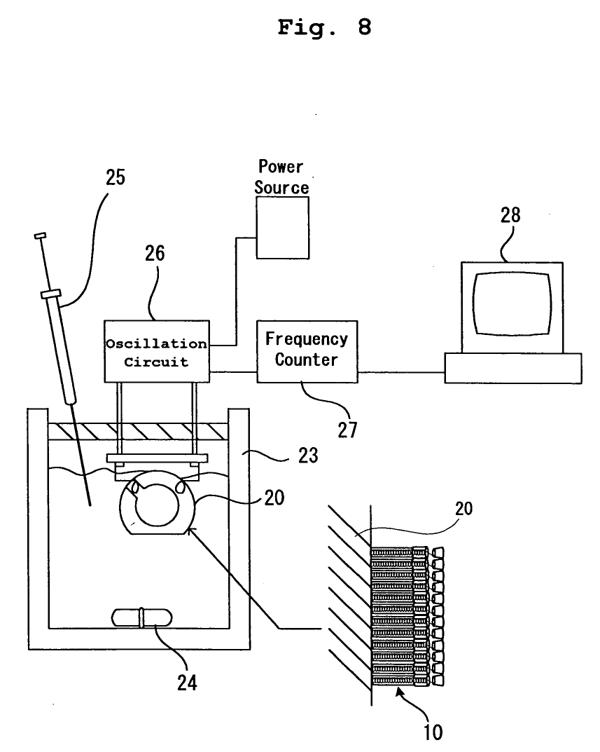

An example of the waste water inspecting apparatus is shown in FIG. 8. The quartz oscillator 20 (waste water inspecting agent 10 is bonded on the surface in a film-like shape) is attached to an arm for attaching the quartz oscillator and dipped in a solution in a thermostat heat block 23. The thermostat heat block 23 is to keep the temperature of the solution constant. The solution is stirred by a stirrer 24. In a sample injection 25, a sample to be measured is injected into a solution. In the oscillation circuit 26, alternating current field is applied to the electrodes 12, 14 of the quartz oscillator 20 to oscillate the quartz oscillator 20. Oscillation frequency of the oscillation circuit 26 is counted by a counter 27, analyzed by a computer 28 and mass of the object to be captured in the sample is indicated.

The object to be captured is specifically captured as such by the capturing bonding material of the waste water inspecting agent in which mass of the waste water inspecting agent changes. The change in the mass is detected by the quartz oscillator and converted to frequency and, therefore, when the change in frequency is measured by the frequency counter, the presence or absence of the object to be captured may be specifically inspected.

When a calibration curve is previously prepared using an object to be captured of a known amount, the object to be captured concentration to be detected or quantified in the sample may be detected or quantified.

The surface acoustic wave (SAW) element is an element where a pair of comb-shaped electrodes is set on the surface of the solid and electric signal is converted to a surface acoustic wave (sonic wave transmitting the solid surface, ultrasonic wave), transmitted to the encountering electrode and outputted as electric signal again whereby signal of specific frequency corresponding to the stimulation may be taken out. Ferroelectric a substance such as lithium tantalite and lithium niobate, quartz, zinc oxide thin film, and the like are used as the material therefor.

The SAW is elastic wave which transmits along the surface of the medium and exponentially decreases in the inside area of the medium. In the SAW, the transmitted energy is concentrated on the surface of the medium whereby the changes in the medium surface may be sensitively detected and, as a result of the changes in the mass of the surface, the SAW transmitting velocity changes as same as in the case of quartz oscillator. Usually, SAW transmitting velocity is measured as the changes in oscillation frequency using an oscillation circuit. Changes in the oscillation frequency are given by the following formula.
Δf=(k₁+k₂)f²hρ−k₂f²h[(4μ/Vᵣ²)(λ+μ/λ2μ)]

In the formula, k₁ and k₂ mean constants, h means thickness of the fixed film, ρ means density of the film, λ and μ mean Lame constants of the film and Vᵣ means a SAW transmitting velocity.

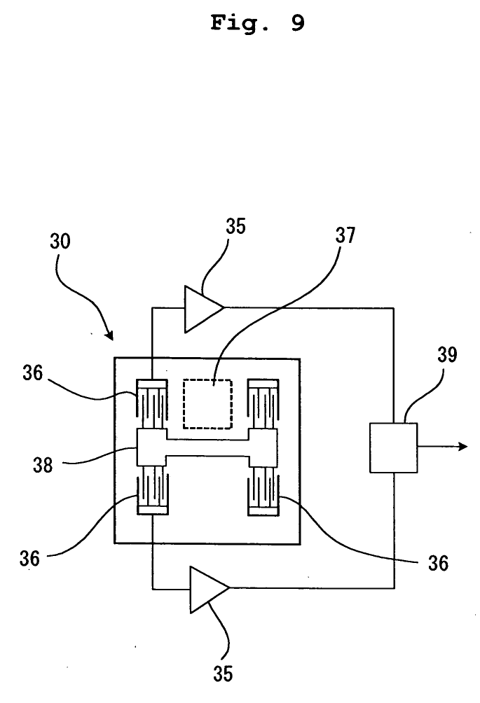

FIG. 9 is a schematic plane view which shows an example of constitution of main parts of a surface acoustic wave (SAW) element. In FIG. 9, in the SAW element sensor 30, there are formed gold electrode 38 and comb-shaped electrodes 36 at both ends thereof on the SAW element having a resonance frequency of 90 MHz made of an ST cut quartz and there is formed a film (not shown) comprising the waste water inspecting agent in the surface wave transmitting region 37 as shown by dotted lines. The sensor is connected to a frequency counter 39 from each comb-shaped electrode 36 via a high-frequency amplifier 35 whereby the mass of the object to be captured in the sample is indicated.

The object to be captured in the sample is specifically captured by the capturing bonding material of the waste water inspecting agent whereby mass or viscoelasticity of the waste water inspecting agent changes, the mass change or viscoelasticity change is detected by the surface acoustic wave (SAW) element and converted to frequency and, therefore, when this frequency change is measured by the frequency counter, it is now possible to specifically examine whether or not the object to be captured is present.

When a calibration curve is previously prepared using an object to be captured of a known amount, the object to be captured to be detected or quantified in the sample may be detected or quantified.

With regard to a method for a chemical bonding/fixing of the waste water inspecting agent on the electrodes of the quartz oscillator or the surface acoustic wave (SAW) element which constitutes the biosensor, there is no particular limitation and that may be appropriately selected according to the object. For example, that may be carried out by means of a chemical bond such as covalent bond.

With regard to the covalent bond method, there is no particular limitation but the same one which is used for bonding the capturing bonding material to the rod-shaped body in the waste water inspecting agent may be appropriately selected and used.

To be specific, there may be exemplified a method where a substance where thiol group is introduced into the end of the waste water inspecting agent is synthesized, the quartz oscillator or the surface acoustic wave (SAW) element is dipped in its solution and made to react therewith for a predetermined time and then the biosensor to which the waste water inspecting agent is chemically bonded/fixed is taken out from the solution followed by drying. The thiol group covers S-trityl-3-mercaptopropyloxy-β-cyanoethyl-N,N-diiso-propylaminophosphoramidide and the like and introduction of the thiol group into the end of the waste water inspecting agent may be carried out by a phosphoramidide method.

### EXAMPLES

Hereinafter, examples of the present invention will be described although the present invention is not limited to those examples at all.

### Example 1

Polymerization of γ-methyl-L-glutamine-N-carboxylic acid anhydride was carried out using tert-butylbenzo-15-crown-5-crown as an initiator to prepare a polypeptide (PMG-CR) having the following formula where tert-butylbenzo-15-crown-5(CR) having a molecule-recognizing ability is located at the molecular chain end.

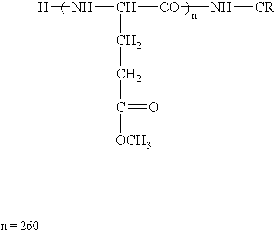

A sample solution containing various metal ions such as alkaline metals (Li, Na, K, and the like), alkaline earth metals (Mg, Ca, and the like) and the like was added to a waste water inspecting agent in which the above polypeptide was emulsified and dispersed and the change in wavelength by the structural color formation was measured using a spectrophotometer in which a significant change in wavelength was observed as compared with the case in which no solution to be inspected was added.

### Example 2

Polymerization of N^{ε}-carbobenzoxy L-lysine N^{α}-carboxylic acid anhydride (LLZ-NCA) was carried out using n-hexylamine as an initiator and then polymerization of γ-methyl L-glutamate N-carboxylic acid anhydride (MLG-NCA) was carried out to prepare a block copolypeptide PLLZ₂₀₀₀-PMLG₆₀₀ where degree of polymerization of a PLLZ moiety was 2000 and that of a PMLG moiety was 600. After that, the PMLG segment was partially hydrolyzed to give L-glutamic acid (LGA) thereby an α-helix copolypeptide PLLZ₂₅₀-P(MLG₄₂₀/LGA₁₈₀) was obtained.

Avidin was introduced into the α-helix copolypeptide and was bonded to a biotin-labeled thioneine-like protein (prepared by a method described in J. Ferment. Bioeny., Vol. 67(4), pages 266-273, 1989) via a biotin-avidin bond to prepare a waste water inspecting agent.

After that, when the waste water inspecting agent was made in a state of being floated (i.e., a horizontal state) on water surface (aqueous phase) and the pH of the water (aqueous phase) was made alkaline of around 12. Consequently, the α-helix structure in the hydrophilic moiety in the waste water inspecting agent was disentangled to give a random structure. At that time, the hydrophobic moiety of the waste water inspecting agent still maintained its α-helix structure. Then, the pH of the water (aqueous phase) was made acidic to approximately 5. As a result, the hydrophilic moiety of the waste water inspecting agent was made into the α-helix structure again. At that time, when the pushing material attached to the waste water inspecting agent was pushed from the side thereof by the pressure of air to the waste water inspecting agent, the hydrophilic moiety was made into the α-helix structure in the direction orthogonal against water surface in the aqueous phase while the waste water inspecting agent was still in a vertical state to the water (aqueous phase). Then, as mentioned above, when the waste water inspecting agent in an aligned state was pushed onto the substrate (plate) using the pushing material, it was possible to form a monomolecular film where the waste water inspecting agent was vertically stood on the substrate (plate). Incidentally, the above operation was carried out using an LB film forming apparatus (NL-LB 400 NK-MWC; manufactured by Nippon Laser & Electronics Laboratories). Thickness of this monomolecular film was calculated to be about 16 nm.

The resulting substrate in which the monomolecular film comprising the waste water inspecting agent was formed was placed in a solution containing a heavy metal (cadmium) and changes in wavelength by structural color formation were measured using a spectrophotometer in which a significant change in wavelength in the polypeptide was observed as compared with a waste water inspecting agent in which the thioneine-like protein was not bonded to the polypeptide.

### Example 3

The monomolecular film where the waste water inspecting agent was vertically formed on the substrate (plate) in Example 2 was used as a constituting unit and it was layered in two layers to prepare a substrate where the waste water inspecting agent was vertically placed in a bimolecular film like form. The substrate was placed in a solution containing a heavy metal (cadmium) and changes in wavelength by structural color formation were measured by a spectrophotometer in which a significant change in wavelength was observed in the polypeptide as compared to the waste water inspecting agent to which the thioneine-like protein was not bonded.

### Example 4

A product in which a gold electrode having an area of 0.2 cm² and a gold-plated lead wire attached to a quartz oscillator (AT cut; area: 0.5 cm²; basic frequency: 9 MHz) was used as a quartz oscillation electrode.

The quartz oscillation electrode was dipped at room temperature for 1 hour in a 1% by volume aqueous solution of aminopropyl triethoxysilane (manufactured by Chisso) and washed by irradiation of ultrasonic wave of 20 kHz in pure water for 30 minutes to remove an excess aminopropyl triethoxysilane. Then, the quartz oscillation electrode was subjected to a thermal treatment for 20 minutes at the temperature of 110° C. whereby a covalent bond was formed between aminopropyl triethoxysilane and quartz oscillator surface.

The quartz oscillator was dipped for 1 hour in a 1% by volume aqueous solution of glutaraldehyde to form a covalent bond between glutaraldehyde and aminopropyl triethoxysilane and, nextly, the quartz oscillator was washed by irradiation with ultrasonic wave of 20 kHz for 30 minutes in pure water to remove an excess glutaraldehyde.

The quartz oscillator electrode was dipped for 2 hours in 100 ml of phosphate buffer of pH 7.2 containing the waste water inspecting agent prepared in Example 2. Consequently, the waste water inspecting agent was fixed to the quartz oscillator via glutaraldehyde. Then, unreacted waste water inspecting agent was removed by washing with a phosphate buffer of pH 7.2.

Then, the quartz oscillator such prepared as was attached to the waste water inspecting apparatus as shown in FIG. 8, a predetermined amount of a solvent containing a heavy metal (cadmium) in a phosphate buffer was added and the changes in frequency during 10 minutes was observed. After one minute, changes in the oscillation frequency nearly reached saturation. An object to which the solvent containing the heavy metal (cadmium) was added showed a significant reduction in oscillation frequency as compared to those without adding such solvent.

It was also observed that, when the added amount of the heavy metal (cadmium) was increased, the oscillation frequency decreased for a certain rate.

### Example 5

A waste water inspecting apparatus was assembled by the same manner as in Example 4 except that a surface acoustic wave (SAW) element of ST cut having oscillation frequency of 10.3 MHz as shown in FIG. 9 was used in place of the quartz oscillator in Example 4.

A predetermined amount of a solvent containing a heavy metal (cadmium) in a phosphate buffer was added thereto and the changes in frequency during 10 minutes were measured. Within one minute, the changes in the oscillation frequency nearly reached saturation. An object to which the solvent containing the heavy metal (cadmium) was added showed a significant reduction in oscillation frequency as compared to those without adding such solvent.

It was also observed that, when the added amount of the heavy metal (cadmium) increased, the oscillation frequency decreased in a certain rate.

In accordance with the present invention, the operation is simple as a whole and it is now possible that harmful heavy metals, cyan compounds, toxic chemical substances, genetic recombinant cells, and the like contained in waste water effluent from factories, and the like may be inspected in quick and easy manner.

1-23. (canceled)

24. A method for inspecting a waste water, comprising:
adding a waste waster inspecting agent to a specimen solution;
aligning the waste water inspecting agent in a film so as to reflect an incident light as a colored interference light,
measuring changes in color tone or wavelength of the colored interference light so as to confirm a presence of an object to be captured in the specimen solution,
wherein the waste water inspecting agent comprises a rod-shaped body and a capturing structured element bonded to the rod-shaped body, and the capturing structured element specifically captures the object.

25. A method for inspecting a waste water according to claim 24, wherein the object to be captured is at least one selected from the group consisting of a heavy metal, a harmful organic compound, an agricultural chemical, an endocrine disruptor in the environment and a genetic recombinant cell.

26. A method for inspecting a waste water according to claim 24, wherein the length of the rod-shaped body is 810 nm or shorter.

27. A method for inspecting a waste water according to claim 24, wherein the rod-shaped body is a helical organic molecule.

28. A method for inspecting a waste water according to claim 27, wherein the helical organic molecule is selected from the group consisting of α-helix polypeptide, DNA and amylose.

29. A method for inspecting a waste water according to claim 24, wherein the waste water inspecting agent is amphiphilic.

30. A method for inspecting a waste water according to claim 24, wherein the capturing structured element is bonded to either an end of the rod-shaped body, or a circumferential side of the rod-shaped body.
